(19) United States
(12) Patent Application Publication
(10) Pub. No.: US 2025/0273334 A1
(43) Pub. Date: Aug. 28, 2025

(54) AI-BASED SYSTEM FOR MEDICAL HISTORY INTERVIEWS, ANALYSIS, AND REPORT GENERATION

(71) Applicant: Christopher Roy Brigham, Hilton Head Island, SC (US)

(72) Inventor: Christopher Roy Brigham, Hilton Head Island, SC (US)

(21) Appl. No.: 19/028,266

(22) Filed: Jan. 17, 2025

Related U.S. Application Data
(60) Provisional application No. 63/558,605, filed on Feb. 27, 2024.

(51) Int. Cl. G16H 50/20; G16H 10/20; G16H 10/60; G16H 15/00

(52) U.S. Cl. CPC G16H 50/20; G16H 10/20; G16H 10/60; G16H 15/00

(57) ABSTRACT

An AI-powered system is disclosed for conducting medical interviews, generating comprehensive medical history reports, and providing clinical insights. The system leverages a large language model (LLM) fine-tuned on domain-specific medical datasets to generate interview prompts, iteratively refine responses based on natural language processing (NLP), and identify incomplete or inconsistent information. The system provides patients with education specific to their situation and tailored to varying levels of health literacy. The system ensures compliance with data protection regulations such as HIPAA and GDPR by employing advanced encryption techniques, multi-factor authentication, and secure data storage. The generated medical history report includes a structured clinical summary, and may include provisional diagnoses with associated ICD-10 codes, clinical analysis, suggestions for diagnostic evaluation and treatment, and other pertinent information. Additional features include EHR integration, customizability, scalability, multilingual support, bias mitigation techniques, and adaptability to diverse demographic groups, ensuring equitable and inclusive healthcare delivery.

### CROSS-REFERENCE TO RELATED APPLICATIONS

The present application claims priority to U.S. Provisional Application No. 63/558,605 filed Feb. 27, 2024, titled “SmartHistory.ai” which is hereby incorporated by reference in its entirety.

### TECHNICAL FIELD

The embodiments generally relate to the technical field of automated history interview and report generation, and in particular, artificial-intelligence-based medical history, analysis and report generation.

### BACKGROUND

Conventional medical history practice may include manually recording and storing a patient's medical history through electronic health record (EHR) systems or, in some cases, paper-based charts. During consultations with patients, providers document key information such as the patient's details, past and current medical conditions, medications, allergies, review of systems, family history, and personal and social habits.

EHR systems enable clinicians to input this data in real time, ensuring accuracy and accessibility. These systems also allow for secure storage, easy retrieval, and sharing of records with other authorized providers to facilitate coordinated care. In environments where paper records are still used, staff manually file them in organized systems, ensuring they are readily available for future visits.

AI-based medical scribing systems interpret conversations that healthcare providers have with their patients; however, these systems passively listen rather than conducting structured interviews with iterative prompts and dynamic refinement using a large language model and analyzing the results.Conventional interview and report-writing systems often result in incomplete documentation and fail to guide critical healthcare issues. Physicians, other health care providers, and professionals often do not have adequate time to obtain adequate histories; therefore, less than optimal care may result.

Careful analysis of information from comprehensive evaluations results in better health outcomes; however, the process of performing an in-depth medical interview, analyzing data, and preparing a report is time-consuming and sometimes unreliable. The same better outcomes apply to other settings where an interview for specific information will result in better management; for example, example, an attorney interviewing a client or a claims professional interviewing a claimant.

The present application claims priority to U.S. Provisional Application No. 63/558,605 filed Feb. 27, 2024, titled “SmartHistory.ai”,” now referred to as “SmartMedHx.ai,” which is hereby incorporated by reference in its entirety.

### SUMMARY

This summary is provided to introduce a variety of concepts in a simplified form that is further disclosed in the detailed description of the embodiments. This summary is not intended to identify key or essential inventive concepts of the claimed subject matter, nor is it intended to determine the scope of the claimed subject matter.

In some aspects, the system includes at least one computing device in operable communication with a network and an application server in operable communication with the user network to host an application program. The application program may be configured for communicating a first interview prompt to a user device, receiving a first response to the first interview prompt; generating a report generation prompt based on the first interview prompt and the first response; receiving a second response to a second report generation prompt in an iterative process; and generating, via an AI-assisted reasoning engine such as a large language model (LLM), a medical history based on the first response and the second response.

In one aspect, an interview module is configured to conduct an interview-style questionnaire with an interviewee user over one or more computer devices operatively connected to one another over a network. The interview may include a plurality of prompts generated by an artificial intelligence (AI) module that has been trained on various interview questions, prompts, medical history data, interviewee-specific data, etc. The interview module may be in operative communication with the AI module to conduct a structured interview to receive user input.

In one aspect, the AI module is configured to intelligently construct a structured interview to receive user input and adjust the structured interview, including altering or adjusting interview questions and prompts, in order to receive user input in a targeted manner. That is, the AI module is configured to use an LLM to perform natural language processing of both interview questions and user input to recognize when a user provides answers to questions that are incorrect or cannot be correct, and the AI module rephrases questions accordingly. In one aspect, the AI module generates feedback based on the user input and generates a case summary for a user to review and provide input such that the AI module may revise the case summary to ensure accuracy.

In one aspect, a report module is configured to generate a report or a report generation prompt based on interview prompts and user responses. The report may be, for example, a medical history based on user responses to interview prompts.

Other illustrative variations within the scope of the invention will become apparent from the detailed description provided hereinafter. The detailed description and enumerated variations, while disclosing optional variations, are intended for purposes of illustration only and are not intended to limit the scope of the invention.

### BRIEF DESCRIPTION OF THE DRAWINGS

A more complete understanding of the embodiments and the attendant advantages and features thereof will be more readily understood by references to the following detailed description when considered in conjunction with the accompanying drawings wherein:

FIG. 1 illustrates a system architecture diagram according to some embodiments;

FIG. 2 illustrates an application program and modules in communication with the computing system, according to some embodiments;

FIG. 3 illustrates a block diagram of a portion of an AI-based medical interview, analysis, and report (MIAR) system, according to some embodiments;

FIG. 4 illustrates a block diagram of a portion of an AI-based MIAR system, according to some embodiments;

FIG. 5 illustrates a block diagram of a portion of an AI-based MIAR system, according to some embodiments;

FIG. 6 illustrates a block diagram of a portion of an AI-based MIAR system, according to some embodiments;

FIG. 7 illustrates a block diagram of a portion of an AI-based MIAR system, according to some embodiments; and

FIG. 8 illustrates a flowchart of a method of implementing an AI-based MIAR system, according to some embodiments.

### DETAILED DESCRIPTION

The specific details of the single embodiment or variety of embodiments described herein are set forth in this application. Any specific details of the embodiments described herein are used for demonstration purposes only, and no unnecessary limitation(s) or inference(s) are to be understood or imputed therefrom.

Before describing exemplary embodiments in detail, it is noted that the embodiments reside primarily in combinations of components related to devices and systems. Accordingly, the device components have been represented where appropriate by conventional symbols in the drawings, showing only those specific details that are pertinent to understanding the embodiments of the present disclosure so as not to obscure the disclosure with details that will be readily apparent to those of ordinary skill in the art having the benefit of the description herein.

The disclosed system may include an AI-assisted reasoning engine, which may include largel language models (LLMs), for tasks such as deduplication, categorization, and contextual interpretation of data, configured for conducting patient interviews and generating comprehensive medical histories and reports based on interview prompts and responses. The system provides a user-centric platform designed to empower users in their healthcare journey. Assigned by a doctor or another professional, the system allows patients to conduct interviews through a software application, such as a smartphone app or web-based platform, providing a detailed account of their medical history and concerns. Upon concluding the interview with a simple “end” command, the data is analyzed based on patient data and data accessed by the AI-assisted reasoning engine, and a comprehensive summary of the patient history and analysis is automatically generated and forwarded to a doctor. In this way, the system simplifies the process of updating medical records. The data analysis utilizes the patient information and information accessible to the AI-assisted reasoning engine to provide insights and recommendations. It ensures that healthcare providers have immediate access to the most current information, facilitating informed decision-making and best practices for personalized care. The analysis provides information that may include, but is not limited to, provisional diagnoses with associated ICD-10 coes, timelines, clinical discussion, recommendations on diagnostic evaluation, treatment, and other case issues, and patient insights.

The system addresses the complex needs of medical history and data management through a secure, accessible, and efficient platform designed for Health Insurance Portability and Accountability Act (HIPPAS) and General Data Protection Regulation (GDPR) compliance with end-to-end encryption and secure data storage and access. The system fosters a secure collaborative environment where admins, team users, and patients work together towards a streamlined and enhanced healthcare experience.

Implementations of the invention involve the technical field of AI-based MIAR, medical scribe, and other computer-based systems, including communicating, via a computing device, a first interview prompt to a user device; generating, via the computing device, a report generation prompt based on the first interview prompt and the first response; and generating, via the computing device implementing a large language model, a medical history report and analysis based on the first response and the second response, and are therefore necessarily rooted in computer technology. In this way, the steps are inherently computer-based because they dynamically refine interview prompts using an AI-based MIAR to address incomplete or inconsistent user data, going beyond static questionnaires associated with typical EHRs. The system is also configured to employ natural language understanding and report and prompt generation in multiple languages, enabling inclusive healthcare delivery.

These cannot be performed in the human mind because they employ an AI-assisted reasoning engine employing NLP of interview prompts and responses strategically to analyze health issues and to generate a medical history report. The present invention amounts to more than merely implementing the generic computer as a tool to gather, analyze, and output data because the steps of the present method, system, or product provide a technical improvement in the field of AI-based EHR systems, including training and re-training an AI-assisted reasoning engine on interview prompts and user responses in order to effectuate an AI-assisted reasoning engine capable of conducting patient interviews kindly, efficiently, with secure storage, easy data retrieval, and the capacity to share records with other authorized providers to facilitate coordinated care. Additionally, the steps of the present invention would be impossible to accomplish on pen and paper due to the volume of data being communicated and received over a network in real-time. In particular, the speed at which the steps of the present invention occur to effectuate the disclosed method, system, or product would involve large-scale, continuous wireless communication of such data. That is, the steps of the present method, system, or product are impossible to accomplish on pen and paper, cannot be accomplished as a method of organizing human activity, and amount to significantly more than merely gathering, analyzing, and outputting data.

Implementations of the present invention include implementing (executing, running, or deploying) one or more artificial intelligence models employing an AI-assisted reasoning engine employing, for example, an advanced large language model, such as Claude 3.5 Sonnet, on a computing device wherein the computing device executes the artificial intelligence model's algorithms and mathematical functions on computer hardware using machine learning libraries. The computing device implements the artificial intelligence model when it performs tasks like training, making predictions, applying the model to data, decision-making, classification, or generating outputs based on user responses to interview questions. In particular, the speed at which an artificial intelligence model analyzes and transforms data to effectuate the disclosed method, system, or product would involve large-scale, continuous transformation of such data. As such, the present invention would be impossible to accomplish on pen and paper or in the human mind due to the volume of data being analyzed and transformed by the artificial intelligence model.

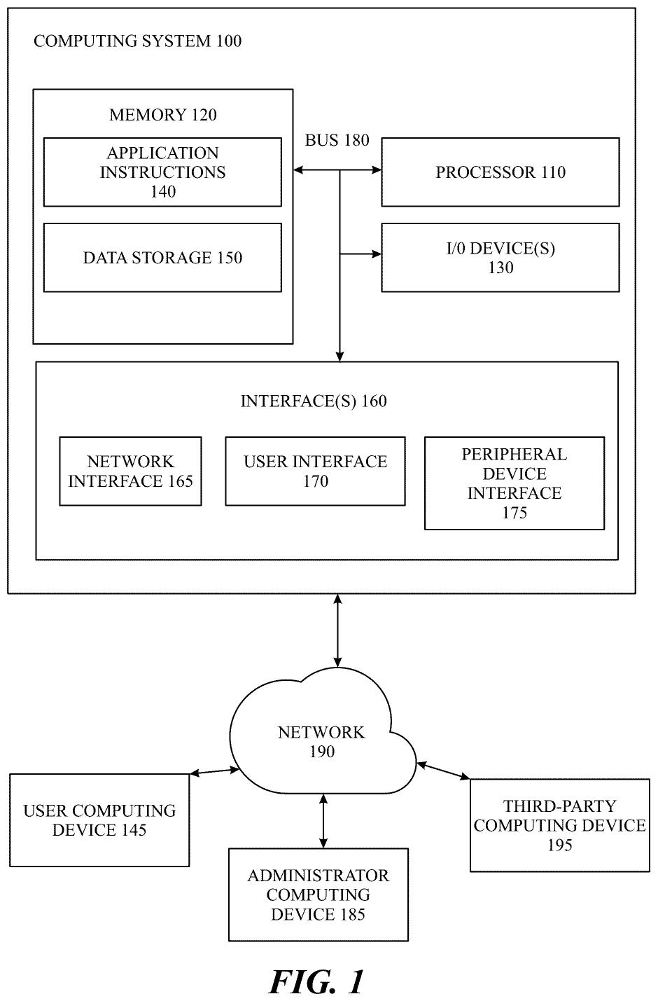

FIG. 1 illustrates an example of a computer system 100 that may be utilized to execute various procedures, including the processes described herein. The computer system 100 comprises a standalone computer or mobile computing device, a mainframe computer system, a workstation, a network computer, a desktop computer, a laptop, or the like. The computer system 100 can be embedded in another device, e.g., a mobile telephone, a personal digital assistant (PDA), a mobile audio or video player, a game console, a Global Positioning System (GPS) receiver, or a portable storage device (e.g., a universal serial bus (USB) flash drive).

In some embodiments, the computer system 100 includes one or more processors 110 coupled to a memory 120 through a system bus 180 that couples various system components, such as an input/output (I/O) devices 130, to the processors 110. The bus 180 may be any of several types of bus structures including a memory bus or memory controller, a peripheral bus, and a local bus using any of a variety of bus architectures. For example, such architectures include Industry Standard Architecture (ISA) bus, Micro Channel Architecture (MCA) bus, Enhanced ISA (EISA) bus, Video Electronics Standards Association (VESA) local bus, and Peripheral Component Interconnect (PCI) bus, also known as Mezzanine bus.

In some embodiments, the computer system 100 includes one or more input/output (I/O) devices 130, such as video device(s) (e.g., a camera), audio device(s), and display(s) are in operable communication with the computer system 100. In some embodiments, similar I/O devices 130 may be separate from the computer system 100 and may interact with one or more nodes of the computer system 100 through a wired or wireless connection, such as over a network interface.

Processors 110 suitable for the execution of computer readable program instructions include both general and special purpose microprocessors and any one or more processors of any digital computing device. For example, each processor 110 may be a single processing unit or a number of processing units and may include single or multiple computing units or multiple processing cores. The processor(s) 110 can be implemented as one or more microprocessors, microcomputers, microcontrollers, digital signal processors, central processing units, state machines, logic circuitries, and/or any devices that manipulate signals based on operational instructions. For example, the processor(s) 110 may be one or more hardware processors and/or logic circuits of any suitable type specifically programmed or configured to execute the algorithms and processes described herein. The processor(s) 110 can be configured to fetch and execute computer readable program instructions stored in the computer-readable media, which can program the processor(s) 110 to perform the functions described herein.

In this disclosure, the term “processor” can refer to substantially any computing processing unit or device, including single-core processors, single-processors with software multithreading execution capability, multi-core processors, multi-core processors with software multithreading execution capability, multi-core processors with hardware multithread technology, parallel platforms, and parallel platforms with distributed shared memory. Additionally, a processor can refer to an integrated circuit, an application specific integrated circuit (ASIC), a digital signal processor (DSP), a field programmable gate array (FPGA), a programmable logic controller (PLC), a complex programmable logic device (CPLD), a discrete gate or transistor logic, discrete hardware components, or any combination thereof designed to perform the functions described herein. Further, processors can exploit nano-scale architectures, such as molecular and quantum-dot based transistors, switches, and gates, to optimize space usage or enhance performance of user equipment. A processor can also be implemented as a combination of computing processing units.

In some embodiments, the memory 120 includes computer-readable application instructions 140, configured to implement certain embodiments described herein, and a database 150, comprising various data accessible by the application instructions 140. In some embodiments, the application instructions 140 include software elements corresponding to one or more of the various embodiments described herein. For example, application instructions 140 may be implemented in various embodiments using any desired programming language, scripting language, or combination of programming and/or scripting languages (e.g., Android, C, C++, C #, JAVA, JAVASCRIPT, PERL, etc.).

In this disclosure, terms “store,” “storage,” “data store,” data storage,” “database,” and substantially any other information storage component relevant to operation and functionality of a component are utilized to refer to “memory components,” which are entities embodied in a “memory,” or components comprising a memory. Those skilled in the art would appreciate that the memory and/or memory components described herein can be volatile memory, nonvolatile memory, or both volatile and nonvolatile memory. Nonvolatile memory can include, for example, read only memory (ROM), programmable ROM (PROM), electrically programmable ROM (EPROM), electrically erasable ROM (EEPROM), flash memory, or nonvolatile random access memory (RAM) (e.g., ferroelectric RAM (FeRAM). Volatile memory can include, for example, RAM, which can act as external cache memory. The memory and/or memory components of the systems or computer-implemented methods can include the foregoing or other suitable types of memory.

Generally, a computing device will also include or be operatively coupled to receive data from or transfer data to, or both, one or more mass data storage devices; however, a computing device need not have such devices. The computer readable storage medium (or media) can be a tangible device that can retain and store instructions for use by an instruction execution device. The computer readable storage medium can be, for example, an electronic storage device, a magnetic storage device, an optical storage device, an electromagnetic storage device, a semiconductor storage device, or any suitable combination of the foregoing. A non-exhaustive list of more specific examples of the computer readable storage medium can include: a portable computer diskette, a hard disk, a random access memory (RAM), a read-only memory (ROM), an erasable programmable read-only memory (EPROM or Flash memory), a static random access memory (SRAM), a portable compact disc read-only memory (CD-ROM), a digital versatile disk (DVD), a memory stick, a floppy disk, a mechanically encoded device such as punch-cards or raised structures in a groove having instructions recorded thereon, and any suitable combination of the foregoing. In this disclosure, a computer readable storage medium is not to be construed as being transitory signals per se, such as radio waves or other freely propagating electromagnetic waves, electromagnetic waves propagating through a waveguide or other transmission media (e.g., light pulses passing through a fiber-optic cable), or electrical signals transmitted through a wire.

In some embodiments, the steps and actions of the application instructions 140 described herein are embodied directly in hardware, in a software module executed by a processor, or in a combination of the two. A software module may reside in RAM, flash memory, ROM memory, EPROM memory, EEPROM memory, registers, a hard disk, a removable disk, a CD-ROM, or any other form of storage medium known in the art. An exemplary storage medium may be coupled to the processor 110 such that the processor 110 can read information from, and write information to, the storage medium. In the alternative, the storage medium may be integrated into the processor 110. Further, in some embodiments, the processor 110 and the storage medium may reside in an Application Specific Integrated Circuit (ASIC). In the alternative, the processor and the storage medium may reside as discrete components in a computing device. Additionally, in some embodiments, the events or actions of a method or algorithm may reside as one or any combination or set of codes and instructions on a machine-readable medium or computer-readable medium, which may be incorporated into a computer program product.

In some embodiments, the application instructions 140 for carrying out operations of the present disclosure can be assembler instructions, instruction-set-architecture (ISA) instructions, machine instructions, machine dependent instructions, microcode, firmware instructions, state-setting data, configuration data for integrated circuitry, or either source code or object code written in any combination of one or more programming languages, including an object oriented programming language such as Smalltalk, C++, or the like, and procedural programming languages, such as the “C” programming language or similar programming languages. The application instructions 140 can execute entirely on the user's computer, partly on the user's computer, as a stand-alone software package, partly on the user's computer and partly on a remote computer, or entirely on the remote computer or server. In the latter scenario, the remote computer can be connected to the user's computer through any type of network, including a local area network (LAN) or a wide area network (WAN), or the connection can be made to an external computer (for example, through the Internet using an Internet Service Provider). In some embodiments, electronic circuitry including, for example, programmable logic circuitry, field-programmable gate arrays (FPGA), or programmable logic arrays (PLA) can execute the computer readable program instructions by utilizing state information of the computer readable program instructions to personalize the electronic circuitry, in order to perform aspects of the present disclosure.

In some embodiments, the application instructions 140 can be downloaded to a computing/processing device from a computer readable storage medium, or to an external computer or external storage device via a network 190. A network adapter card or network interface in each computing/processing device receives computer readable program instructions from the network and forwards the computer readable application instructions 140 for storage in a computer readable storage medium within the respective computing/processing device.

In some embodiments, the computer system 100 includes one or more interfaces 160 that allow the computer system 100 to interact with other systems, devices, or computing environments. In some embodiments, the computer system 100 comprises a network interface 165 to communicate with a network 190. In some embodiments, the network interface 165 is configured to allow data to be exchanged between the computer system 100 and other devices attached to the network 190, such as other computer systems, or between nodes of the computer system 100. In various embodiments, the network interface 165 may support communication via wired or wireless general data networks, such as any suitable type of Ethernet network, for example, via telecommunications/telephony networks such as analog voice networks or digital fiber communications networks, via storage area networks such as Fiber Channel SANs, or via any other suitable type of network and/or protocol. Other interfaces include the user interface 170 and the peripheral device interface 175.

In some embodiments, the network 190 corresponds to a local area network (LAN), wide area network (WAN), the Internet, a direct peer-to-peer network (e.g., device to device Wi-Fi, Bluetooth, etc.), and/or an indirect peer-to-peer network (e.g., devices communicating through a server, router, or other network device). The network 190 can comprise copper transmission cables, optical transmission fibers, wireless transmission, routers, firewalls, switches, gateway computers and/or edge servers. The network 190 can represent a single network or multiple networks. In some embodiments, the network 190 used by the various devices of the computer system 100 is selected based on the proximity of the devices to one another or some other factor. For example, when a first user device and second user device are near each other (e.g., within a threshold distance, within direct communication range, etc.), the first user device may exchange data using a direct peer-to-peer network. But when the first user device and the second user device are not near each other, the first user device and the second user device may exchange data using a peer-to-peer network (e.g., the Internet). The Internet refers to the specific collection of networks and routers communicating using an Internet Protocol (“IP”) including higher level protocols, such as Transmission Control Protocol/Internet Protocol (“TCP/IP”) or the Uniform Datagram Packet/Internet Protocol (“UDP/IP”).

Any connection between the components of the system may be associated with a computer-readable medium. For example, if software is transmitted from a website, server, or other remote source using a coaxial cable, fiber optic cable, twisted pair, digital subscriber line (DSL), or wireless technologies such as infrared, radio, and microwave, then the coaxial cable, fiber optic cable, twisted pair, DSL, or wireless technologies such as infrared, radio, and microwave are included in the definition of medium. As used herein, the terms “disk” and “disc” include compact disc (CD), laser disc, optical disc, digital versatile disc (DVD), floppy disk, and Blu-ray disc; in which “disks” usually reproduce data magnetically, and “discs” usually reproduce data optically with lasers. Combinations of the above should also be included within the scope of computer-readable media. In some embodiments, the computer-readable media includes volatile and nonvolatile memory and/or removable and non-removable media implemented in any type of technology for storage of information, such as computer-readable instructions, data structures, program modules, or other data. Such computer-readable media may include RAM, ROM, EEPROM, flash memory or other memory technology, optical storage, solid state storage, magnetic tape, magnetic disk storage, RAID storage systems, storage arrays, network attached storage, storage area networks, cloud storage, or any other medium that can be used to store the desired information and that can be accessed by a computing device. Depending on the configuration of the computing device, the computer-readable media may be a type of computer-readable storage media and/or a tangible non-transitory media to the extent that when mentioned, non-transitory computer-readable media exclude media such as energy, carrier signals, electromagnetic waves, and signals per se.

In some embodiments, the system is world-wide-web (www) based, and the network server is a web server delivering HTML, XML, etc., web pages to the computing devices. In other embodiments, a client-server architecture may be implemented, in which a network server executes enterprise and custom software, exchanging data with custom client applications running on the computing device.

In some embodiments, the system can also be implemented in cloud computing environments. In this context, “cloud computing” refers to a model for enabling ubiquitous, convenient, on-demand network access to a shared pool of configurable computing resources (e.g., networks, servers, storage, applications, and services) that can be rapidly provisioned via virtualization and released with minimal management effort or service provider interaction, and then scaled accordingly. A cloud model can be composed of various characteristics (e.g., on-demand self-service, broad network access, resource pooling, rapid elasticity, measured service, etc.), service models (e.g., Software as a Service (“SaaS”), Platform as a Service (“PaaS”), Infrastructure as a Service (“IaaS”), and deployment models (e.g., private cloud, community cloud, public cloud, hybrid cloud, etc.).

As used herein, the term “add-on” (or “plug-in”) refers to computing instructions configured to extend the functionality of a computer program, where the add-on is developed specifically for the computer program. The term “add-on data” refers to data included with, generated by, or organized by an add-on. Computer programs can include computing instructions, or an application programming interface (API) configured for communication between the computer program and an add-on. For example, a computer program can be configured to look in a specific directory for add-ons developed for the specific computer program. To add an add-on to a computer program, for example, a user can download the add-on from a website and install the add-on in an appropriate directory on the user's computer.

In some embodiments, the computer system 100 may include a user computing device 145, an administrator computing device 185 and a third-party computing device 195 each in communication via the network 190. The user computing device 145 may be utilized by a user to interact with the various functionalities of the system, including registration, login, viewing user records, participating in questionnaires or interviews provided by the system, etc. The administrator computing device 185 is utilized by an administrative user to moderate content and to perform other administrative functions. The third-party computing device 195 may be utilized by third parties to receive communications from the user computing device, transmit communications to the user via the network, and otherwise interact with the various functionalities of the system, such as via application program interface, web portal, etc.

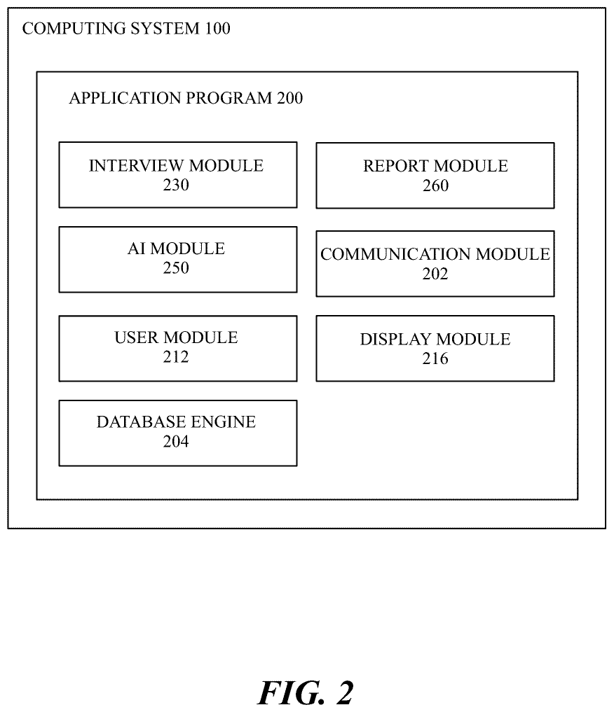

FIG. 2 illustrates an example computer architecture for the application program 200 operated via the computing system 100. The computer system 100 comprises several modules and engines configured to execute the functionalities of the application program 200, and a database engine 204 configured to facilitate how data is stored and managed in one or more databases. In particular, FIG. 2 is a block diagram showing the modules and engines needed to perform specific tasks within the application program 200.

Referring to FIG. 2, the computing system 100 operating the application program 200 comprises one or more modules having the necessary routines and data structures for performing specific tasks, and one or more engines configured to determine how the platform manages and manipulates data. In some embodiments, the application program 200 comprises one or more of an interview module 230, a report module 260, an AI module 250, a communication module 202, a database engine 204, a user module 212, and a display module 216.

In some embodiments, the interview module 230 is configured to conduct an interview-style history-taking with an interviewee user over one or more computer devices operatively connected to one another over a network, such as via the user computing device 145, third-party computing device 195, and network 190 of FIG. 1. The interview may include a plurality of prompts generated by the AI module 250 that has been trained on various interview questions, prompts, medical history data, interviewee-specific data, etc. The type of interview performed is dependent on the situation. With the use of AI, text is converted to voice, and voice is converted to text for processing. In some embodiments, the AI module 250 is configured to generate additional interview prompts that mimic human expression, via text or voice in one or more languages. With the use of AI, interviews may be performed in multiple languages. In some embodiments, the interview uses rule-based logic to provide prompts based on user input. The interview module 230 may be in operative communication with the AI module 250 to conduct a structured interview in order to receive user input to prompts. For example, the interview module 230 may incorporate a chatbot configured to conduct an interview, including prompts provided by the AI module 250. That is, the interview module 230 may leverage the AI module 250 to use natural language processing (NLP), and programmed rules to simulate multi-lingual human-like conversations, by voice or text, with a user participating in an interview. When a user inputs a text message into the interview, the chatbot processes the text using NLP or similar technique to interpret its meaning and intent. When a user interacts via voice, text content generated via the LLM is converted to voice, and voice responses are converted to text for NLP or similar technique to interpret its meaning and intent. NLP may involve breaking the message into components, analyzing the context, and identifying keywords or patterns. Based on this analysis, the interview module 230 selects an appropriate response from its pre-programmed database or generates a new response using algorithms employed by the AI module 250. In embodiments, the interview module 230 continuously improves by learning from interactions with users, adapting its prompts and responses to become more accurate and contextually relevant over time. In this way, the interview module 230 is configured to collect user responses to prompts and responses may be stored or used to re-train the AI module 250. The LLM is trained and fine-tuned for medical interviews to ensure completeness and accuracy, and to avoid hallucationations. The interview may include, as a non-limiting example, polite greetings to establish rapport, questions to establish an accurate and complete history, such as, to assess clinical, functional impairment, and medicolegal issues, and to clarify incomplete or potentially inaccurate responses. Prompts may address patient medical history present and past, prescription and over-the-counter medications, family medical history, short and long term ailments, personal and social history and occupational history etc. Prompts, and responses to prompts, may be saved and stored, such as via the database engine 204, and may be used to train or re-train the AI module 250 to increase interview efficacy.

In some embodiments, the AI module 250 is configured to intelligently construct a structured interview to receive user input and adjust the structured interview, including altering or adjusting interview questions and prompts, in order to receive user input in a targeted manner. That is, the AI module may be configured to use an AI-assisted reasoning engine which may include an LLM to perform natural language processing of both interview questions and user input to recognize when a user provides answers to questions that are incorrect or cannot be correct, and the AI module rephrases questions accordingly. An AI-assisted reasoning engine may include computer-based algorithm(s) designed to analyze data, draw inferences, and solve problems by leveraging artificial intelligence techniques. In embodiments, the AI module 250 may perform tokenization, part-of-speech tagging, dependency parsing, intent recognition, and entity extraction to predict responses or interview questions dynamically. In embodiments, the AI module 250 may be rule-based and may rely on a predefined set of rules and decision trees to match user inputs with responses. In embodiments, the AI module 250 uses a machine learning model to predict responses dynamically. The AI module 250 may be trained on datasets of conversation examples from existing medical reports, for example, enabling the AI module 250 to generate contextually relevant and diverse replies, including reducing bias. That is, the AI module 250 may employ constitutional artificial intelligence principles to avoid bias, which may include fundamental guidelines designed to ensure the development and deployment of AI systems align with ethical, legal, and societal norms such as transparency, fairness, non-discrimination, safety, privacy, etc. In still other embodiments, the AI module 250 may employ deep learning, such as transformer-based system architectures, to generate responses that mimic human language and adapt to nuanced user inputs. In some embodiments, the AI module 250 may communicate in multiple languages via text or voice. The AI module 250 may also incorporate additional data from APIs or databases if a user query or response requires external information. In this way, the AI module 250 is configured to generate report-generation prompts based on interview prompts and responses, receive additional responses to additional prompts, and generate a medical history based on the responses to questions that make up an interview.

In embodiments, the AI module 250 is trained on both a plurality of predefined interview prompts as well as user responses to the same. Additionally, the AI module 250 is trained on known medical conditions, diseases, etc. that are not specific to any user but are specific to a potential diagnosis associated with a user being interviewed. Similarly, the AI module 250 may be continuously re-trained on new prompts, user responses, or medical information in order to improve the accuracy and efficacy of the AI module 250.

In some embodiments, the report module 260 is configured to map responses received by the interview module 230 to predefined fields of a report, such as a medical history report. Responses may be mapped to predefined fields, such as by, but not limited to, field mapping of database columns to reports fields, data transformation, field population, or document generation, such as converting the report to a specific document format. In this way, the report module 260 accurately and efficiently maps user responses to the appropriate fields of a report, thereby generating a medical history based on user responses.

In some embodiments, the communication module 202 is configured for receiving, processing, and transmitting a user command and/or one or more data streams. In such embodiments, the communication module 202 performs communication functions between various devices, including the user computing device 145 of FIG. 1, the administrator computing device 185 of FIG. 1, and a third-party computing device 195 of FIG. 1. In some embodiments, the communication module 202 is configured to allow one or more users of the system, including a third-party, to communicate with one another. In some embodiments, the communications module 202 is configured to maintain one or more communication sessions with one or more servers, the administrative computing device 185 of FIG. 1, and/or one or more third-party computing device(s) 195 of FIG. 1. In some embodiments, the communication module 202 may allow users and administrators to communicate with one another.

In some embodiments, a database engine 204 is configured to facilitate the storage, management, and retrieval of data to and from one or more storage mediums, such as the one or more internal databases described herein. In some embodiments, the database engine 204 is coupled to an external storage system. In some embodiments, the database engine 204 is configured to apply changes to one or more databases. In some embodiments, the database engine 204 comprises a search engine component for searching through thousands of data sources stored in different locations. Database engine 204 may be configured to store prompts, user response, generated reports, AI training data, etc.

The user module 212 may store user preferences including the user account information, historical usage data, user personal information, and the like. The user module 212 may facilitate the creation of user's profiles for users, administrators, and others.

In some embodiments, the display module 216 is configured to display one or more graphic user interfaces, including, e.g., one or more user interfaces. In some embodiments, the display module 216 is configured to temporarily generate and display various pieces of information in response to one or more commands or operations. The various pieces of information or data generated and displayed may be transiently generated and displayed, and the displayed content in the display module 216 may be refreshed and replaced with different content upon the receipt of different commands or operations in some embodiments. In such embodiments, the various pieces of information generated and displayed in a display module 216 may not be persistently stored. The display module 216 displays information, notifications, and alerts to the user device which can be viewed and acknowledged by the user. In some embodiments, based on the information provided by the patient, an AI-assisted reasoning engine, which may include an LLM, may provide education to the patient, e.g., information about the medical condition(s), health and wellness, tailored to the level of health literacy.

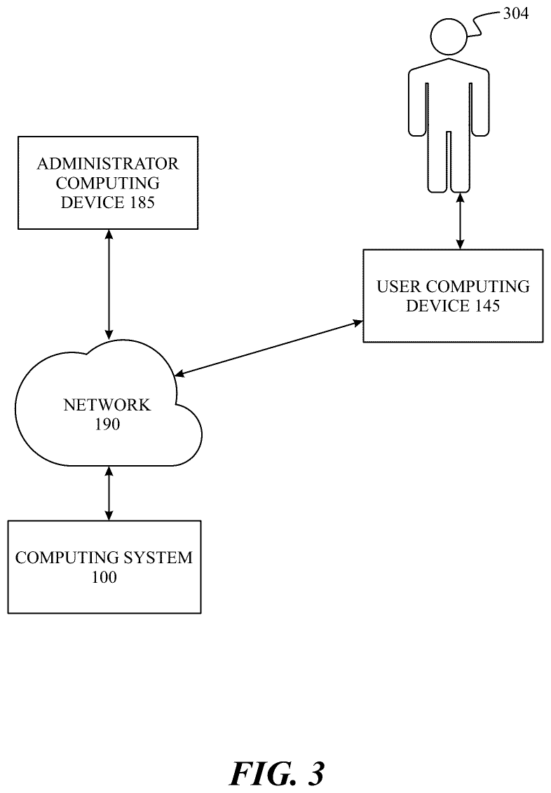

FIG. 3 illustrates a method of interacting with AI-based AI-based medical interview, analysis, and report (MIAR) system, depicted as computing system 100, similar to computing system 100 of FIGS. 1 and 2, which may be in operable communication with a user computing device 145 and an administrator computing device 185 over network 190. Computing system 100 may communicate with a user computing device 145 such that a user 304 may participate in an interview as described herein.

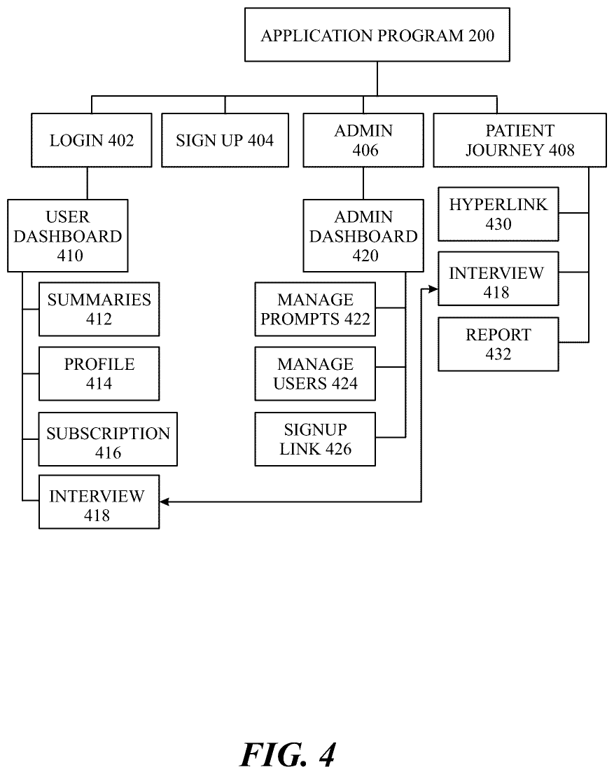

FIG. 4 illustrates a block diagram of a portion of an AI-based MIAR system, including the application program 200 as described with respect to FIGS. 1 and 2, and which is executed by the computing system of FIGS. 1 through 3. The application program 200 may include functionality for sign up 404, login 402, admin 406, such as an administrator user, and patient journey 408. Sign up 404 may include a user interface or dashboard for receiving new user information to utilize the system. This may include name, age, medical history, username, password, etc. Sign-up 404 may be directly related to login 402 in that, after completion of sign-up 404, a user may log in 402 and be presented with a user dashboard 410 on a device, such as a user computing device 145 of FIG. 3. User dashboard 410 may be a user interface including menus, clickable links, etc. that allow a user to access interview summaries (reports) 412, user profile details 414, system subscription 416 information, and interview 418. Interview 418 may include the previously described interview or questionnaire facilitated by the application program 200, and specifically the interview module 230, report module 260, and AI module 250 of FIG. 2. In use, patient journey 408 may include a user requesting interview 418. In response, the application program 200 generates a hyperlink 430 to interview 418, which the user may participate in. Upon completion of interview 418, report 432 may be generated via the report module 260 of FIG. 2. The report 432 may be distributed to a computing device of a physician, another health care provider, professional, individual, or patient and, in some embodiments, a verbal or audio history of the report 432 is generated. In some embodiments, hyperlink 430 may have a timed expiration or may expire upon completion of the interview 418.

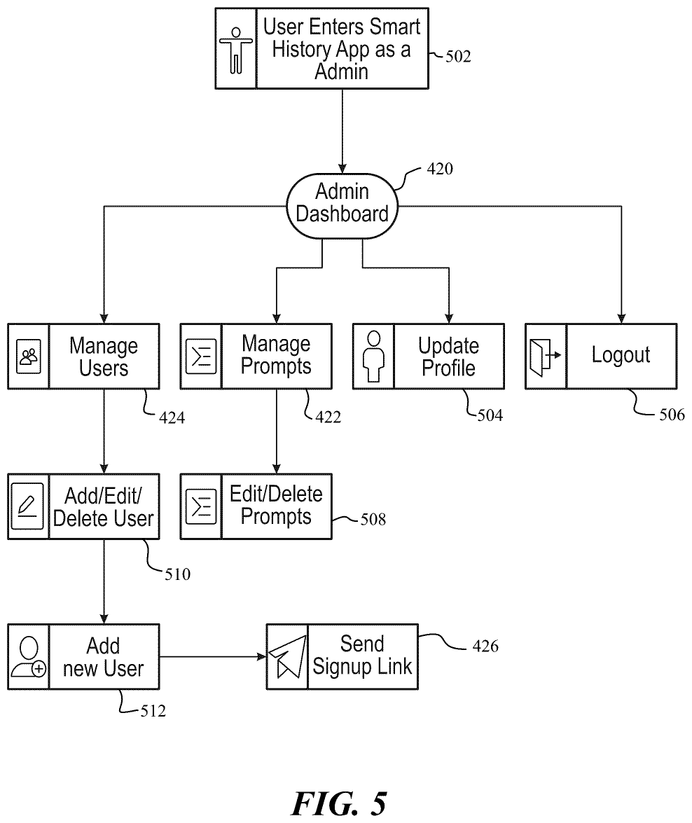

FIG. 5 illustrates a block diagram of a portion of an AI-based MIAR system including an admin dashboard 420 associated with admin 406 of FIG. 4. Admin 406 may access the system, i.e. application program 200 as described with respect to FIGS. 1 and 2, as an administrator 502 to manage users 424, manage prompts 422 used by the interview module 230, report module 260, and AI module 250 of FIG. 2, update profiles 504, or logout 506 of the system. Managing users 424 may include adding, editing, or deleting users 510, including adding users 512 and sending signup links 426, such as via email. Managing prompt 422 may include manual addition, edit, or deletion of prompt 508 from a database, such as database engine 204 of FIG. 2, that the interview module 230, report module 260, and AI module 250 of FIG. 2 rely upon to generate interview prompts. In this way, admins 406 may manually tailor prompts as needed to effectuate an improved interview process.

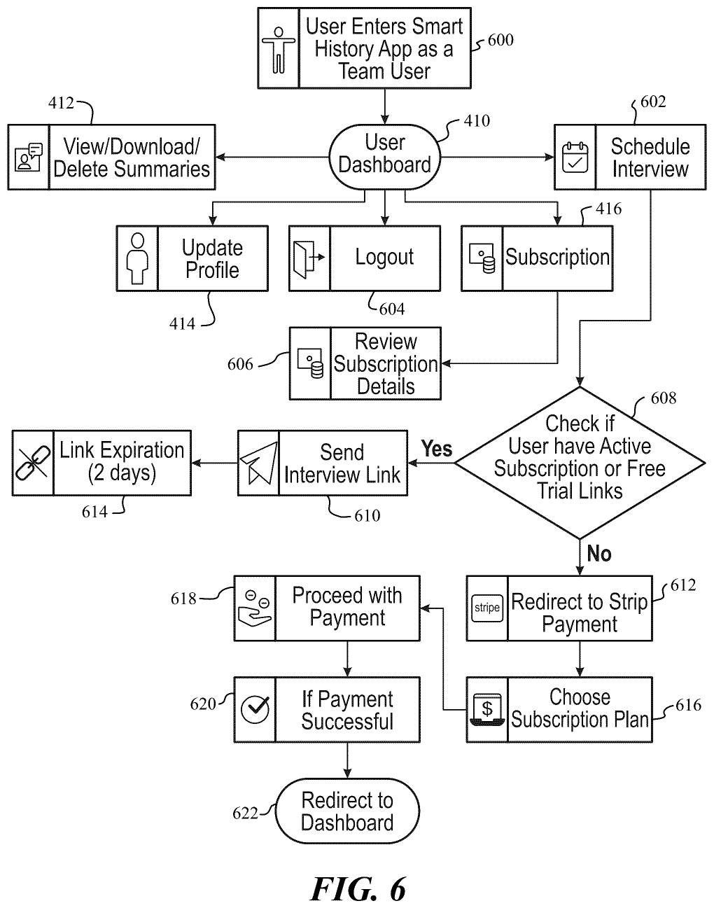

FIG. 6 illustrates a block diagram of a portion of an AI-based MIAR system including a user dashboard 410 corresponding to the user dashboard 410 of FIG. 4. A user may access the system, i.e. the “Smart Medical History App,” as a team user (i.e. an administrator) to view, download, or delete summaries (reports) 412 or schedule interviews 602. Scheduling an interview 602 may including confirming 608 that a patient (interviewee) user is authorized to be interviewed, such as by submitting a free trial link or has paid for a subscription to the system. If yes, the team user may select the type of interview and language, and specifies the name and contact information (email or phone number for text messaging), and an interview link may be sent 610 to the patient user. In some embodiments, the link may automatically expire 614, such as after five days. If the team user does not have credits for scheduling interviews the system may redirect the user's UI to a payment portal 612, permit the user to sign up for a subscription plan 616, submit payment 618, verify payment 620, and redirect 622 the user UI to the user dashboard 410 of FIG. 4. Team users may also be able to review subscription 416 details 606 including modifying payment terms, pricing plans, update user profile details 414, etc.

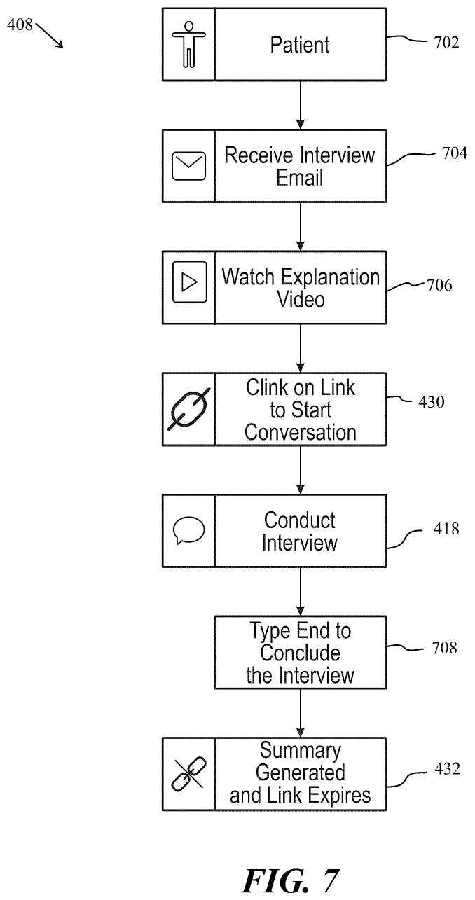

FIG. 7 illustrates a block diagram of a portion of an AI-based MIAR system, including patient journey 408, also depicted in FIG. 4. In patient journey 408, a patient 408 702 may receive an interview email 704, including a hyperlink to an interview generated by the system. The email may also include an introductory or explanation video 706 about the interview process. The patient 702 may click the hyperlink 430, depicted as hyperlink 430 in FIG. 4, to begin the interview. The patient 702 may interact with interview 418 corresponding to interview 418 of FIG. 4. In some embodiments the patient may receive feedback based on the information they provide. Upon completion, patient 702 may type “end” in the interview response window to conclude 708 the interview. In response, the system may generate a report 432 corresponding to report 432 of FIG. 4, and expire the hyperlink 430, depicted as hyperlink 430 in FIG. 4.

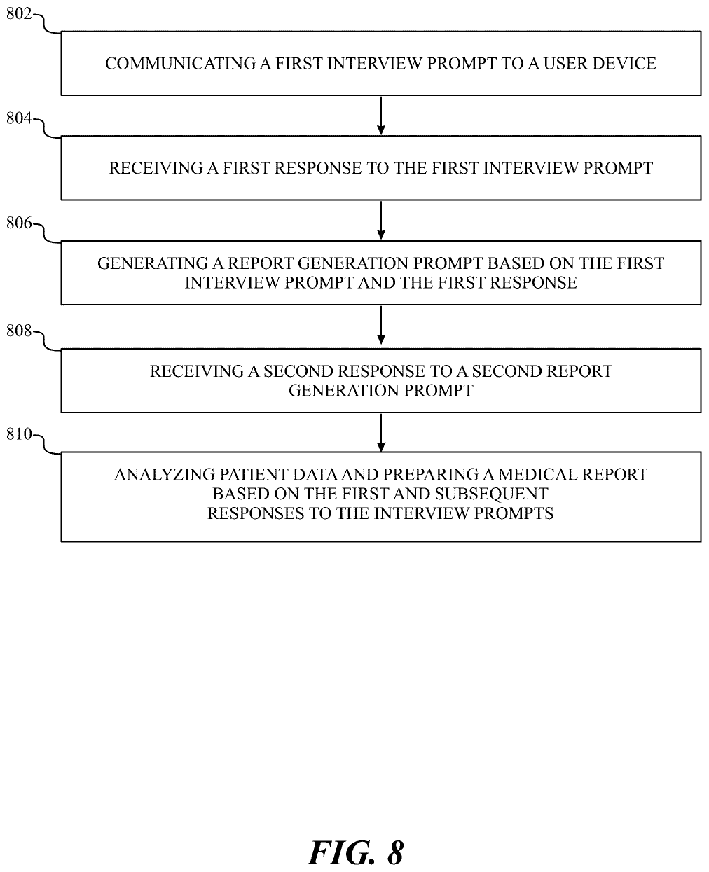

FIG. 8 illustrates a flowchart of a method of implementing an AI-based MIAR system, including, in step 802, communicating, via a computing device, a first interview prompt to a user device via the interview module 230 of FIG. 2. Step 804 may include receiving, as input to the user device, a first response to the first interview prompt via the interview module 230 of FIG. 2. Step 806 may include generating, via the computing device, a report generation prompt based on the first interview prompt and the first response via the interview module 230 and the AI module 250 of FIG. 2. Step 808 may include receiving, as input to the user device, a second response to a second report generation prompt and subsequent iterative prompts via the interview module 230 of FIG. 2. This process is repeated until the interview is complete, i.e, the process is iterative. Step 810 may include analyzing patient data and preparing a report based on the first and subsequent responses to the interview prompts via the report module 260 of FIG. 2.

In this disclosure, the various embodiments are described with reference to the flowchart illustrations and/or block diagrams of methods, apparatus (systems), and computer program products. Those skilled in the art would understand that each block of the flowchart illustrations and/or block diagrams and combinations of blocks in the flowchart illustrations and/or block diagrams can be implemented by computer-readable program instructions. The computer-readable program instructions can be provided to a processor of a general purpose computer, special purpose computer, or other programmable data processing apparatus to produce a machine, such that the instructions, which execute via the processor of the computer or other programmable data processing apparatus, create means for implementing the functions or acts specified in the flowchart and/or block diagram block or blocks. The computer-readable program instructions can be stored in a computer-readable storage medium that can direct a computer, a programmable data processing apparatus, and/or other devices to function in a particular manner, such that the computer-readable storage medium having instructions stored therein comprises an article of manufacture including instructions which implement aspects of the function/act specified in the flowchart and/or block diagram block or blocks. The computer-readable program instructions can be loaded onto a computer, other programmable data processing apparatus, or other devices to cause a series of operational acts to be performed on the computer, other programmable apparatus, or other devices to produce a computer-implemented process, such that the instructions that execute on the computer, other programmable apparatus, or other device implement the functions or acts specified in the flowchart and/or block diagram block or blocks.

In this disclosure, the block diagrams in the Figures illustrate the architecture, functionality, and operation of possible implementations of systems, methods, and computer program products according to the various embodiments. Each block in the flowchart or block diagrams can represent a module, segment, or portion of instructions, which comprises one or more executable instructions for implementing the specified logical function(s). In some embodiments, the functions noted in the blocks can occur out of the order noted in the Figures. For example, two blocks shown in succession can, in fact, be executed concurrently or substantially concurrently, or the blocks can sometimes be executed in the reverse order, depending upon the functionality involved. In some embodiments, each block of the block diagrams and/or flowchart illustration, and combinations of blocks in the block diagrams and/or flowchart illustration, can be implemented by a special purpose hardware-based system that performs the specified functions or acts or carries out combinations of special purpose hardware and computer instructions.

In this disclosure, the subject matter has been described in the general context of computer-executable instructions of a computer program product running on a computer or computers, and those skilled in the art would recognize that this disclosure can be implemented in combination with other program modules. Generally, program modules include routines, programs, components, data structures, etc., that perform particular tasks and/or implement particular abstract data types. Those skilled in the art would appreciate that the computer-implemented methods disclosed herein can be practiced with other computer system configurations, including single-processor or multiprocessor computer systems, mini-computing devices, mainframe computers, as well as computers, hand-held computing devices (e.g., PDA, phone), microprocessor-based or programmable consumer or industrial electronics, and the like. The illustrated embodiments can be practiced in distributed computing environments where tasks are performed by remote processing devices that are linked through a communications network. Some embodiments of this disclosure can be practiced on a stand-alone computer. In a distributed computing environment, program modules can be located in both local and remote memory storage devices.

In this disclosure, the terms “component,” “system,” “platform,” “interface,” and the like, can refer to and/or include a computer-related entity or an entity related to an operational machine with one or more specific functionalities. The disclosed entities can be hardware, a combination of hardware and software, software, or software in execution. For example, a component can be a process running on a processor, a processor, an object, an executable, a thread of execution, a program, and/or a computer. By way of illustration, both an application running on a server and the server can be a component. One or more components can reside within a process and/or thread of execution, and a component can be localized on one computer and/or distributed between two or more computers. In another example, respective components can computer-readablebe executed from various computer-readable media with various data structures stored thereon. The components can communicate via local and/or remote processes, such as in accordance with a signal having one or more data packets (e.g., data from one component interacting with another component in a local system, distributed system, and/or across a network such as the Internet with other systems via the signal). As another example, a component can be an apparatus with specific functionality provided by mechanical parts operated by electric or electronic circuitry, which is operated by a software or firmware application executed by a processor. In such a case, the processor can be internal or external to the apparatus and can execute at least a part of the software or firmware application. As another example, a component can be an apparatus that provides specific functionality through electronic components without mechanical parts, wherein the electronic components can include a processor or other means to execute software or firmware that confers at least in part the functionality of the electronic components. In some embodiments, a component can emulate an electronic component via a virtual machine, e.g., within a cloud computing system.

The phrase “application” as is used herein means software other than the operating system, such as Word processors, database managers, Internet browsers and the like. Each application generally has its own user interface, which allows a user to interact with a particular program. The user interface for most operating systems and applications is a graphical user interface (GUI), which uses graphical screen elements, such as windows (which are used to separate the screen into distinct work areas), icons (which are small images that represent computer resources, such as files), pull-down menus (which give a user a list of options), scroll bars (which allow a user to move up and down a window) and buttons (which can be “pushed” with a click of a mouse). A wide variety of applications is known to those in the art.

The phrases “Application Program Interface” and API, as are used herein, mean a set of commands, functions, and/or protocols that computer programmers can use when building software for a specific operating system. The API allows programmers to use predefined functions to interact with an operating system, instead of writing them from scratch. Common computer operating systems, including Windows, Unix, and the Mac OS, usually provide an API for programmers. An API is also used by hardware devices that run software programs. The API generally makes a programmer's job easier, and it also benefits the end user since it generally ensures that all programs using the same API will have a similar user interface.

The phrases “computing device” or “central processing unit” as is used herein means a computer hardware component that executes individual commands of a computer software program. It reads program instructions from a main or secondary memory, and then executes the instructions one at a time until the program ends. During execution, the program may display information to an output device such as a monitor.

The term “execute” as is used herein in connection with a computer, console, server system, or the like means to run, use, operate, or carry out an instruction, code, software, program, and/or the like.

In this disclosure, the descriptions of the various embodiments have been presented for purposes of illustration and are not intended to be exhaustive or limited to the embodiments disclosed. Many modifications and variations will be apparent to those of ordinary skill in the art without departing from the scope and spirit of the described embodiments. The terminology used herein was chosen to best explain the principles of the embodiments, the practical application or technical improvement over technologies found in the marketplace, or to enable others of ordinary skill in the art to understand the embodiments disclosed herein. Thus, the appended claims should be construed broadly, to include other variants and embodiments, which may be made by those skilled in the art. It will be appreciated by persons skilled in the art that the present embodiment is not limited to what has been particularly shown and described hereinabove. A variety of modifications and variations are possible considering the above teachings without departing from the following claims.

I claim:

1. A computer-implemented method comprising:
communicating, via a computing device, a first interview prompt to a user device;
receiving, as input to the user device, a first response to the first interview prompt;
dynamically generating, via an AI-assisted reasoning enginefine-tuned on domain-specific medical datasets, a second interview prompt based on semantic analysis of the first response, wherein the AI-assisted reasoning enginedetects incomplete or inconsistent data and generates targeted follow-up prompts;
generating a structured medical history report comprising clinical summaries and provisional diagnoses based on the semantic analysis of the first response; and
integrating the structured medical history report into an electronic health record (EHR) systems via standardized interoperability frameworks while ensuring compliance with data protection regulations.

2. The method of claim 1, further comprising securely transmitting the structured medical history report and analysis to a healthcare provider or authorized professional via an encrypted communication channel using AES-256 encryption and multi-factor authentication, wherein access to the report is time-limited and role-based to ensure compliance with HIPAA and GDPR regulations.

3. The method of claim 1, wherein the AI-assisted reasoning engine comprises a large language model configured to mitigate bias and harm by employing adversarial training techniques, fairness-aware algorithms, and continuous monitoring of model outputs to ensure equitable treatment across diverse demographic groups.

4. The method of claim 1, wherein the AI-assisted reasoning engine comprises a large language model that comprises constitutional artificial intelligence principles configured to avoid bias or harm to a user.

5. The method of claim 1, wherein the AI-assisted reasoning engine comprises a large language model that is configured to understand natural language patterns and generate additional interview prompts that mimic human expression, via text or voice in one or more languages.

6. The method of claim 1, further comprising generating and communicating a time-sensitive, encrypted hyperlink to a first interview session, wherein the hyperlink requires multi-factor authentication for access and automatically expires upon completion of the interview or after a predefined time period.

7. The method of claim 7, further comprising expiring, via the computing device, the hyperlink in response to generating the medical history based on the first response and the second response.

8. A computer-implemented method comprising:
communicating, via a computing device, a first interview prompt to a user device receiving, as input to the user device;
a first response to the first interview prompt;
generating, via the computing device implementing a AI-assisted reasoning engine, a report generation prompt based on the first interview prompt and the first response;
receiving, as input to the user device, a second response to a second report generation prompt; and
generating, via the computing device implementing the AI-assisted reasoning engine, an analysis and report based on the first response and the second response with the initial application being a medical report summarizing the history and providing analysis and clinical insights.

9. The method of claim 9, wherein the first interview prompt and the second report generation prompt comprise questions and a report with the initial application being medical.

10. The method of claim 9, further comprising communicating a medical history and the analysis to a health care provider or the report to a person.

11. The method of claim 9, wherein generating the first interview prompt comprises generating the first interview prompt based on rule-based logic with multilingual communication by voice or text.

12. The method of claim 9, wherein the AI-assisted reasoning engine comprises constitutional artificial intelligence principles configured to avoid bias or harm to a user.

13. The method of claim 9, wherein the AI-assisted reasoning engine is configured to understand natural language patterns and generate additional interview prompts that mimic human expression.

14. The method of claim 9, further comprising generating and communicating, via a computing device, a hyperlink to a first interview comprising at least a first interview prompt prior to the communicating.

15. The method of claim 15, further comprising expiring, via the computing device, the hyperlink in response to generating the report in response to the generating the report based on the first response and the second response with an initial application being a report presenting the medical information in a structured fashion with clinial analysis.

16. A software product comprising at least one computer-readable storage media having application instructions collectively stored on the at least one computer-readable storage media, the application instructions executable to:
generate and communicate a hyperlink corresponding to a first interview comprising at least a first interview prompt;
communicate the first interview prompt to a user device;
receive, as input to the user device, a first response to the first interview prompt;
generate, via a large language model, a second interview prompt based on the first interview prompt and the first response;
receive, as input to the user device, a second response to the second interview prompt;
generate, via a large language model, a medical history based on the first response and the second response; and
expire the hyperlink in response to generating the medical history.

17. The software product of claim 17, wherein the first interview prompt and the second interview prompt comprise medical history questions.

18. The software product of claim 17, wherein the large language model comprises constitutional artificial intelligence principles configured to avoid bias or harm to a user.

19. The software product of claim 17, wherein the large language model is configured to understand natural language patterns and generate additional interview prompts that mimic human expression.
